(19) United States
(12) Patent Application Publication
(10) Pub. No.: US 2014/0171888 A1
(43) Pub. Date: Jun. 19, 2014

(54) TRIMMABLE WOUND BANDAGE

(71) Applicant: PAUL HARTMANN AG, Heidenheim (DE)

(72) Inventors: PIERRE CROIZAT, Herbrechtingen (DE); AXEL ECKSTEIN, Heidenheim (DE); JÜRGEN HOFSTETTER, Heidenheim (DE)

(73) Assignee: PAUL HARTMANN AG, Heidenheim (DE)

(21) Appl. No.: 14/107,804

(22) Filed: Dec. 16, 2013

Related U.S. Application Data
(60) Provisional application No. 61/766,447, filed on Feb. 19, 2013.

(30) Foreign Application Priority Data
Dec. 17, 2012 (DE) 10 2012 223 399.7

(51) Int. Cl. A61F 13/02; A61M 1/00

(52) U.S. Cl. CPC A61F 13/0236; A61M 1/0088
USPC 604/319

(57) ABSTRACT

A trimmable wound bandage, in particular film or foil bandage, for air- and negative pressure tight closure of a wound space in negative pressure therapy, includes a flat material layer, in particular on film or foil basis; which effects the air- and negative pressure tight closure, a support element provided on a wound-averted side of the flat material layer and stabilizing the flat material layer in areal extent during application of the bandage, a glue coating provided on a wound-facing side of the flat material layer, a detachable protective layer covering the glue coating until application of the bandage, and multiple manually graspable, in particular tab-shaped or strip-shaped gripping regions provided in circumferential direction of the bandage and distributed over an outer circumference of the bandage, said multiple gripping regions not adhering to the glue coating for facilitating detachment of the protective layer.

### CROSS-REFERENCES TO RELATED APPLICATIONS

This application claims the priority of German Patent Application, Serial No. 10 2012 223 399.7, filed Dec. 17, 2012, pursuant to 35 U.S.C. 119(a)-(d), the disclosure of which is incorporated herein by reference.

This application claims the benefit of prior filed U.S. provisional Application No. 61/766,477, filed Feb. 19, 2013, pursuant to 35 U.S.C. 119(e), the disclosure of which is incorporated herein by reference.

### BACKGROUND OF THE INVENTION

The present invention relates to a trimmable wound bandage.

The following discussion of related art is provided to assist the reader in understanding the advantages of the invention, and is not to be construed as an admission that this related art is prior art to this invention.

During the negative pressure therapy of wounds a negative pressure-generating device communicates with the wound or the wound space via a suction line, wherein an air- and negative pressure-tight wound bandage for air- and negative pressure-tight closure of the wound and the wound space is provided so that a negative pressure can be generated in the wound space and liquids can be suctioned away from the wound space into a collecting container at the negative pressure generating device.

The term negative pressure in the context of the present invention means an air pressure, which is decreased relative to the ambient pressure (atmospheric air pressure), in particular inside a wound bandage. The material of a wound bandage for air- and negative pressure-tight closure of a wound space therefore has to be configured so as to withstand the established pressure differential in order to be able to apply and maintain the negative pressure in the wound space in the first place. In the field of negative pressure therapy for wound treatment, the negative pressure is stated quantitatively as pressure differential between the environmental air pressure and the air pressure applied underneath the cover material. Typically, this pressure differential in the field of negative pressure therapy is at most 250 mm Hg (mm Mercury column) (1 mm Hg=1 Torr.=133,322 Pa). This negative pressure range up to at most 250 mm Hg has proven appropriate for the wound healing. A preferred negative pressure range is between 10 and 250 mm Hg.

The above-mentioned air- and negative pressure-tight closure of the wound space does not mean that no gas exchange between the wound space and its environment may occur. Rather, air- and negative pressure-tight closure in this context means that with regard to utilizing a particular negative pressure generating device, the negative pressure required for the negative pressure therapy of wounds can be generated and maintained in the wound space. Therefore, materials can also be used which are slightly permeable for gas so long as the negative pressure required for the negative pressure therapy can be maintained. Typically, the water vapor permeability of the flat material layer which effects the air- and negative pressure-tight closure is 100 to 2500 g/m^{2·24} h, in particular 500 to 2000 g/m²·24 h, in particular 800 to 1600 g/m²·24 h, in particular 1052 to 1450 g/m²·24 h, determined according to DIN EN 13726—2 at 37° C. and 50% relative humidity.

Due to the complexity of wounds, bandages are often used that can be cut to size and which are cut to size directly on the patient and applied. Oftentimes multiple trimmed bandage sections are also applied on top of each other with more or less overlap in order to achieve a sufficient sealing for the negative pressure therapy, which is often made difficult by unevennesses of the skin, scab etc. In addition, especially poorly healing wounds are often very expansive or irregularly shaped and frequently have a projected surface of greater than 100 cm². This requires bandages that can be cut to size.

Bandages are known which have strip-shaped gripping regions for grasping and detaching the release layer or protective layer from the glue coating; these gripping regions extend in particular centered across the product and are formed by folding back a strip-shaped section of the protective layer onto itself. Further, bandage means in the broadest sense are known with a tab-shaped or gripping strip-shaped gripping region provided on the border. EP 144-0667 A1 discloses a wound cover in which a strip-shaped gripping region is provided on each of two opposing sides.

When cutting a section from a flat, trimmable bandage, the problem often arises that this section does not have a manually graspable gripping region, thus complicating detachment of the protective layer from the glue coating, whereby the air- and negative pressure-tight flat material layer is also often adversely affected.

It would therefore be desirable and advantageous to provide an improved trimmable bandage to obviate these prior art shortcomings.

### SUMMARY OF THE INVENTION

According to one aspect of the present invention, a trimmable wound bandage, in particular film or foil bandage, for air- and negative pressure-tight closure of a wound space in negative pressure therapy includes a flat material layer, in particular on film or foil basis, which effects the air- and negative pressure-tight closure, a support element provided on a wound-averted side of the flat material layer and stabilizing the flat material layer in its areal extent during application of the bandage, a glue coating provided on a wound-facing side of the flat material layer, a detachable protective layer covering the glue coating until application of the bandage; and manually graspable, in particular tab-shaped or strip-shaped gripping regions provided in circumferential direction of the bandage distributed over the outer circumference, which do not adhere on the glue coating for facilitating detachment of the protective layer.

This ensures for almost all situations in which a flat bandage is cut to size that at least one graspable region is present in the section that has been separated from the bandage and is to be applied, thereby enabling detachment of the protective layer from the glue coating without adversely affecting the flat material layer which effects the air and negative pressure tight closure. Overall, this enables a very user-friendly and in particular time-saving application of the bandage. In addition, the amount of bandage material that is rendered unusable as a result of non-detachable protective layer can be reduced.

According to another advantageous feature of the invention, the plurality of gripping regions includes in particular at least 5, in particular at least 8, in particular at least 10 and further in particular at least 12 gripping regions, which are distributed along the circumference of the wound bandage.

The gripping regions may be formed within the geometric circumferential border of the bandage in that the protective layer and the flat material layer are non-adhesively interconnected at this location. On the other hand it is advantageous when the gripping regions are configured so as to protrude transverse to the circumferential direction of the bandage, i.e., so as to protrude over a circumferential border which delimits the actual bandage, because in this way they can be manually gripped more easily.

According to another advantageous feature of the invention, the gripping regions may be configured integrally as one piece with the protective layer, i.e., they are formed by loop-, tab-, web- or strip-shaped attachments or protrusions of the protective layer. However, they can also be formed by loop-, tab-, web- or strip-shaped attachments which are attached to the protective layer or are inserted between the protective layer and the flat material layer.

It is advantageous that in a manner of speaking gripping regions are provided so as to be distributed over the entire outer circumference of the bandage. In a further refinement of the invention is proposed that the distance of the gripping regions to each other in circumferential direction of the bandage measured along a border edge is at least 10 mm, in particular at least 15 mm, in particular at least 20 mm, in particular at least 25 mm, and at most 120 mm, in particular at most 100 mm, in particular most 80 mm, in particular at most 60 mm.

The support element, which is more bending stiff and torsion stiff compared to the sealing flat material layer, is intended to hold and stabilize the rather thin, film- or foil-like flat material layer in its areal extent, so as to facilitate application of the bandage on the body surface of the patient in the intended manner. A configuration of the support element, which the support element covers the entire areal extent of the sealing, flat material layer on the other hand is problematic because this adversely affects the three-dimensional drapability of the bandage. In this context it is optimal when the support element is provided, so as to enframe the bandage or bandage section to be applied.

A further problem that often arises in bandages of the type discussed here during cutting to size, i.e., during separation of sections from the bandage to be applied, is that the flat material layer of the separated section has a lower stability due to the fact that the support element is then only present to a decreased degree for example only along one or two borders of the separated section.

According to another advantageous feature of the invention, the support element can sections, which extend strip-shaped or web-shaped inwardly from an outer circumference region of the bandage. Strip-shaped or web-shaped extending sections can meet and can in particular cross over. In this way an improved stabilization of the bandage and sections separated from the bandage can be realized because the support element covers a greater area of the sealing flat material sections in the predominant portion of cut configurations.

According to another advantageous feature of the invention the support element can have multiple weakening lines, in particular perforations, which extend transverse to the extent of the support element along an outer circumferential region and/or transverse to the extent of its strip- or web-shaped extending sections. At these weakening lines, the support element can easily be manually grasped and, after applying the bandage onto the body surface, be detached from the sealing flat material layer.

According to another advantageous feature of the invention the weakening lines can be spaced apart by at least 30 mm, in particular at least 40 mm, in particular at least 50 mm, in particular at least 60 mm, and that most 150 mm, in particular at most 130 mm, in particular most 100 mm. this distance in case of doubt is measured along a radially outer border edge of the actual bandage in circumferential direction of the bandage.

According to another advantageous feature of the invention, the flat material layer, which effects the air- and negative pressure-tight closure can be configured transparent. Preferably it is formed on the basis of a water-insoluble polymer or on the basis of a metal foil or metallized polymer foil. Its thickness is preferably 10 μm to 500 μm, in particular 25 μm to 100 μm. The water insoluble polymer has a solubility of ≦10 mg/l, in particular of ≦1 mg/l, in particular of 0.0001 to 1 mg/l, determined according to the column elution method according to EU-guideline RL 67-548-EWG, Appendix V, chapter A6. The flat material layer is in particular formed on polyurethane-, polyester-, polypropylene-, polyethylene-, polyamide-, polyvinyl chloride-, or polyorganosiloxane (silicone) basis or from mixtures thereof.

According to another advantageous feature of the invention, the glue coating of the flat material layer can be configured grid-shaped in the broadest sense. With regard to the intended sealing effect, however, it is advantageous when the glue coating is applied to the flat material layer over its entire surface. It is currently preferred when the glue coating is formed on acrylate basis. Preferably, the glue coating includes a pressure sensitive adhesive (PSA). However, it is also conceivable to use a so-called switchable glue which for example is activated by UV irradiation.

According to another advantageous feature of the invention, the detachable protective layer which covers the glue coating can be formed on paper- or polymer film-basis, in particular on polyethylene-, polypropylene- or polyester-basis.

According to another advantageous feature of the invention, the detachable protective layer can have an anti-adhesive coating in order to enable an essentially resistance-free detachability of the dense flat material layer. This anti-adhesive coating is provided on one or both surfaces of the detachable protective layer. Typically, it provides this surface with hydrophobic or lipophobic properties and is for example silicone-, fluorocarbon- or oil-based.

The aforementioned support element is advantageously formed on paper- or polymer film-basis, in particular on polyethylene-, polypropylene-, polyester- or polyamide-basis. The support element has advantageously a thickness of 10-500 μm, in particular of 20 to 250 μm, in particular of 25-100 μm. The support element may also have a anti-adhesive coating on its side which faces the flat material layer so that the support element can be easily detached after application of the bandage to the body surface of patient without exerting excessive peeling or shearing forces onto the flat material layer.

According to another advantageous feature of the invention, the composite of sealing flat material layer and carrier element is generated in that the flat material layer is directly extruded onto a side of the support element. The support element is also preferably produced in the form of a continuous layer, i.e., an areally continuously extending layer, wherein then recesses are later formed in the support element layer by way of special cutting techniques (“kiss cut”) without adversely affecting the sealing flat material layer.

### BRIEF DESCRIPTION OF THE DRAWING

Other features and advantages of the present invention will be more readily apparent upon reading the following description of currently preferred exemplified embodiments of the invention with reference to the accompanying drawing, in which:

FIG. 1 shows a top view onto a bandage according to the invention that can be cut to size; and

FIG. 2 shows a schematic layer sequence as sectional view with section plane II-II in FIG. 1.

### DETAILED DESCRIPTION OF PREFERRED EMBODIMENTS

Throughout all the Figures, same or corresponding elements are generally indicated by same reference numerals. These depicted embodiments are to be understood as illustrative of the invention and not as limiting in any way. It should also be understood that the drawings are not necessarily to scale and that the embodiments are sometimes illustrated by graphic symbols, phantom lines, diagrammatic representations and fragmentary views. In certain instances, details which are not necessary for an understanding of the present invention or which render other details difficult to perceive may have been omitted.

The Figures show a trimmable wound bandage overall designated with the reference numeral 2, for use during negative pressure therapy for air- and negative pressure-tight closure of a not shown wound space. The negative pressure communication of a negative pressure generating device with the wound space is accomplished by passing a conducting means, typically and preferably by using a not shown connection device, through a through-opening that formed in the bandage. The trimmable bandage 2 shown in the Figures can be configured very expansively. In particular, the bandage 2 can have any longitudinal or transverse dimensions, and is configured so that sections of substantially any desired geometry can be cut from the bandage 2, in order to be used for negative pressure-tight covering and closing of a wound space, wherein the wound bandage or the wound bandage sections are adhesively fastened on undamaged skin surfaces around the wound.

The wound bandage 2 includes a flat material layer 4 which effects the actual air- and negative pressure-tight closure, a support element 8 which is provided on the wound-averted side 6 of the flat material layer 4 and in the exemplary case forms frame, a glue coating 10 on a wound-facing side 12 of the flat material layer 4, and a detachable protective layer 14 which covers the glue coating 10. In addition gripping regions 16 can be seen which do not adhere to the glue coating 10, and can be manually grasped and facilitate the detachment of the protective layer 14 from the glue coating 10.

In the exemplary and preferred shown case, the support element is configured to form a frame, i.e., it extends in a circumferential direction 18 along an outer border region or outer circumferential region 20, preferably continuously. This ensures an areal stabilization of the flat material layer 4, which compared to the support element 8 is rather areally unstable, in particular “flimsy”. The support element 8 or its material is more bending stiff than the sealing flat material layer 4 or its material and therefore is capable of stabilizing the flat material layer 4.

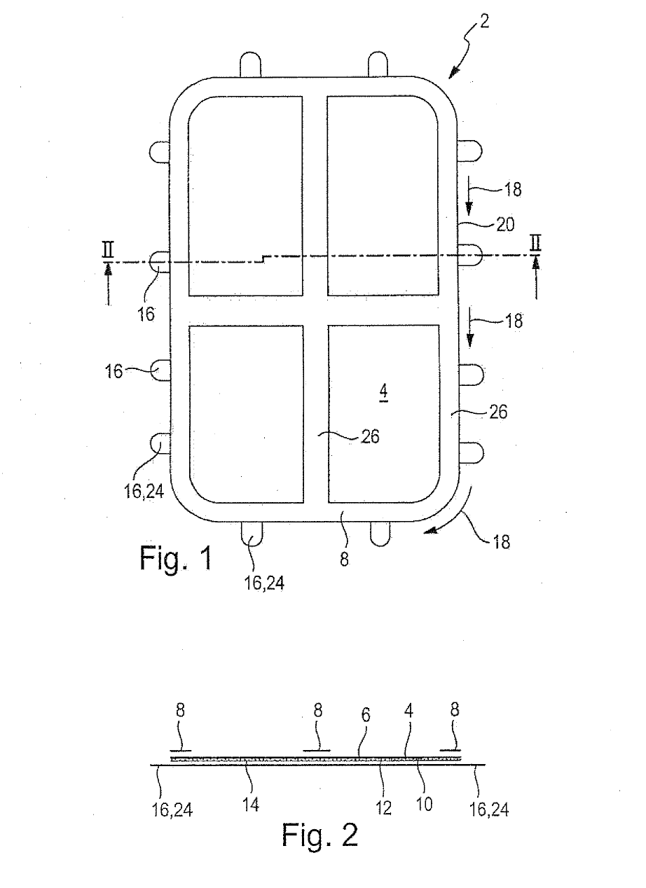

The support element 8 also includes sections 22 which extend strip-shaped or web-shaped inwardly from the outer border region 20, and in the exemplary shown case meet each other and thereby cross over each other. This achieves a further stabilization of the flat material layer 4 during application. When a more or less large section is cut off from the bandage 2 shown in FIG. 1, the support element 8 is not only provided along the outer border region 20 but at least portions of the inwardly extending sections 22 of the support element 8 additionally contribute to the stabilization. This separated section of the bandage is therefore significantly better stabilized compared to an embodiment without these inwardly and strip-shaped or web-shaped extending sections 22. It is noted that the geometry exemplary shown in. FIG. 1 is not strictly required and numerous deviations or other embodiments of sections that extend strip- or web-shaped inwardly are conceivable and advantageous.

The support element 8 is provided detachably adherent on the wound-averted side 6 of the sealing flat material layer 4. It can detachably adhere on the flat material layer 4 for example by mechanical pressing, by heat lamination, by means of a glue layer or by static interactions. After applying the flat material layer 4 on the patient, the support element 8 is removed from the body-averted side 6 of the flat material layer 4.

The wound bandage further includes a plurality of gripping regions 16, which are provided in succession distributed over the outer circumference in circumferential direction 18. The gripping regions 16 protrude outwardly transverse to the circumferential direction 18. In the exemplary shown case, these gripping regions 16 are formed as outwardly protruding loops or tabs 24 which are formed integral and as one piece with the protective layer 14. It is also possible, however, that the gripping regions 16 are formed by material sections that are separate from the protective layer 14, and are attached to the protective layer 14 or are inserted between the flat material layer 4 and the protective layer 14. It is also conceivable that multiple gripping regions are provided in circumferential direction 18 distributed over the outer circumference, so as to not protrude outwardly but situated within a circumferential contour. In this case it is also advantageous when the arrangement of the gripping regions is marked in order to enable an easier, more targeted engagement with a finger. Because multiple gripping regions 16 are provided in succession in circumferential direction 18, even a separated section of the trimmable bandaged 2 shown in FIG. 1 includes such a gripping region 16 with high likelihood, which ensures that the protective layer 14 in each separated section can be easily detached.

According to another advantageous feature of the invention, the support element 8 can include multiple weakening lines 26, in particular in form of perforations which extend transverse to the respective extent of the support element 8. After applying the sealing flat material layer 4 on the patient it is advantageous that in the region of these weakening lines the support element 26 can be lifted relatively easily from the wound-averted side 6 of the sealing flat material layer 4 and then be detached as a whole. It is also conceivable and advantageous when the support element 8 has manually graspable and preferably outwardly protruding gripping regions. These gripping regions may be arranged so as to completely or partially overlap with the gripping regions assigned to the protective layer.

In the exemplary shown case, the trimmable bandage 2 is shaped rectangular. At each of its respective opposing straight borders, two or four gripping regions are for example provided, The rectangular shape of the trimmable bandage 2 is purely exemplary and expedient; other configurations, in particular round or oval configurations, are also conceivable. For the respective configuration, dimensioning and arrangement of the flat material layer 4, the support element 8, the glue coating 10, the protective layer 14 and the gripping region 16, the same considerations apply as above.

While the invention has been illustrated and described in connection with currently preferred embodiments shown and described in detail, it is not intended to be limited to the details shown since various modifications and structural changes may be made without departing in any way from the spirit of the present invention. The embodiments were chosen and described in order to best explain the principles of the invention and practical application to thereby enable a person skilled in the art to best utilize the invention and various embodiments with various modifications as are suited to the particular use contemplated.

What is claimed as new and desired to be protected by Letters Patent is set forth in the appended claims and includes equivalents of the elements recited therein:

What is claimed is:

1. A trimmable wound bandage, in particular film or foil bandage, for air- and negative pressure-tight closure of a wound space in negative pressure therapy, comprising:
a flat material layer, in particular on film or foil basis, which effects the air- and negative pressure-tight closure;
a support element provided on a wound-averted side of the flat material layer and stabilizing the flat material layer in areal extent during application of the wound bandage;
a glue coating provided on a wound-facing side of the flat material layer;
a detachable protective layer covering the glue coating until application of the bandage; and
multiple manually graspable, in particular tab-shaped or strip-shaped gripping regions provided in circumferential direction of the bandage and distributed over an outer circumference of the bandage, said multiple gripping regions configured to not adhere to the glue coating for facilitating detachment of the protective layer.

2. The bandage of claim 1, wherein the support element forms a frame.

3. The bandage of claim 1, wherein the gripping regions are configured to protrude transverse to the circumferential direction of the bandage.

4. The bandage of claim 1, wherein the gripping regions are configured integral as one piece with the protective layer.

5. The bandage of claim 1, wherein the gripping regions are formed by one of loop-, tab-web, or strip-shaped attachments attached to the protective layer or inserted between the protective layer and the flat material layer.

6. The bandage of claim 1, wherein a distance of the gripping regions to each other in circumferential direction of the bandage measured along a border edge of the bandage is at least 10 mm, in particular at least 15 mm, in particular at least 20 mm, in particular at least 25 mm, and at most 120 mm, in particular at most 100 mm, and particularly at most 80 mm, in particular at most 60 mm.

7. The bandage of claim 1, wherein the support element has sections which extend strip- or web-shaped inwardly from an outer circumferential region of the bandage.

8. The bandage of claim 7, wherein the strip or web shaped extending sections meet each other and in particular cross over each other.

9. The bandage of claim 1, wherein the support element has multiple weakening lines, in particular perforations which extend transverse to its extent along an outer circumference region and/or transverse to the extent of its strip or web shaped extending sections.

10. The bandage of claim 9, wherein the weakening lines are spaced apart from each other by at least 30 mm, in particular at least 40 mm, in particular at least 50 mm, in particular at least 60 mm, and at most 150 mm, a particular at most 130 mm, in particular at most 100 mm.

11. The bandage of claim 1, wherein the flat material layer is configured transparent.

12. The bandage of claim 1, wherein the flat material layer is formed on the basis of at least one member selected from the group of polyurethane, polyester, polypropylene polyethylene, polyamide, polyvinyl chloride, and polyorganisiloxan.

13. The bandage of claim 1, wherein the glue coating of the flat material layer is applied over its entire surface.

14. The bandage of claim 1, wherein the glue coating of the flat material layer is formed on acrylate basis.

15. The bandage of claim 1, wherein the detachable protective layer is formed on paper-basis or polymer film-basis, in particular on polyethylene-, polypropylene- or polyester basis.

16. The bandage of claim 1, wherein the detachable protective layer has an anti-adhesive coating.

17. The bandage of claim 1, wherein the support element is formed on paper-basis or polymer film-basis, in particular on polyethylene-, polypropylene-, polyester- or polyamide-basis.

18. The bandage of claim 1, wherein the support element has a thickness of 10-250 μm, in particular 25-100 μm.

19. The bandage of claim 1, wherein the support element has an anti-adhesive coating on a side which faces the flat material layer.

20. The bandage of claim 1, wherein the flat material layer is extruded directly onto a side of the support element.
